(19) United States
(12) Patent Application Publication
(10) Pub. No.: US 2021/0024928 A1
(43) Pub. Date: Jan. 28, 2021

(54) C/EBP ALPHA SARNA COMPOSITIONS AND METHODS OF USE

(71) Applicant: MiNA THERAPEUTICS LIMITED, London (GB)

(72) Inventors: Vikash Reebye, London (GB); David Blakey, London (GB)

(21) Appl. No.: 16/970,090

(22) PCT Filed: Feb. 15, 2019
(86) PCT No.: PCT/EP2019/053872

§ 371 Date: Aug. 14, 2020

Related U.S. Application Data
(60) Provisional application No. 62/631,583, filed on Feb. 16, 2018.

(51) Int. Cl. C12N 15/113; A61K 9/127

(52) U.S. Cl. CPC C12N 15/113; A61K 9/127

(57) ABSTRACT

The invention relates to saRNA targeting a C/EBPα transcript and therapeutic compositions comprising said saRNA. Methods of using the therapeutic compositions are also provided.

### CROSS-REFERENCE TO RELATED APPLICATIONS

This application claims priority to U.S. Prov. Application No. 62/631,583 filed Feb. 16, 2018, the contents of which are incorporated herein by reference in their entirety.

### SEQUENCE LISTING

The present application is being filed along with a Sequence Listing in electronic format. The sequence listing filed, entitled 20581022PCT_SL.txt, was created on Feb. 15, 2019 and is 6,635 bytes in size. The information in electronic format of the Sequence Listing is incorporated herein by reference in its entirety.

### FIELD OF THE INVENTION

The invention relates to polynucleotide, specifically saRNA, compositions for modulating C/EBPα and C/EBPα pathways and to the methods of using the compositions in therapeutic applications.

### BACKGROUND OF THE INVENTION

CCAAT/enhancer-binding protein α (C/EBPα, C/EBP alpha, C/EBPA or CEBPA) is a leucine zipper protein that is conserved across humans and rats. This nuclear transcription factor is enriched in hepatocytes, myelomonocytes, adipocytes, as well as other types of mammary epithelial cells [Lekstrom-Himes et al., J. Bio. Chem, vol. 273, 28545-28548 (1998)]. It is composed of two transactivation domains in the N-terminal part, and a leucine zipper region mediating dimerization with other C/EBP family members and a DNA-binding domain in the C-terminal part. The binding sites for the family of C/EBP transcription factors are present in the promoter regions of numerous genes that are involved in the maintenance of normal hepatocyte function and response to injury. C/EBPα has a pleiotropic effect on the transcription of several liver-specific genes implicated in the immune and inflammatory responses, development, cell proliferation, anti-apoptosis, and several metabolic pathways [Darlington et al., Current Opinion of Genetic Development, vol. 5(5), 565-570 (1995)]. It is essential for maintaining the differentiated state of hepatocytes. It activates albumin transcription and coordinates the expression of genes encoding multiple ornithine cycle enzymes involved in urea production, therefore playing an important role in normal liver function.

In the adult liver, C/EBPα is defined as functioning in terminally differentiated hepatocytes whilst rapidly proliferating hepatoma cells express only a fraction of C/EBPα [Umek et al., Science, vol. 251, 288-292 (1991)]. C/EBPα is known to up-regulate p21, a strong inhibitor of cell proliferation through the up-regulation of retinoblastoma and inhibition of Cdk2 and Cdk4 [Timchenko et al., Genes & Development, vol. 10, 804-815 (1996); Wang et al., Molecular Cell, vol. 8, 817-828 (2001)]. In hepatocellular carcinoma (HCC), C/EBPα functions as a tumor suppressor with anti-proliferative properties [Iakova et al., Seminars in Cancer Biology, vol. 21(1), 28-34 (2011)].

Different approaches are carried out to study C/EBPα mRNA or protein modulation. It is known that C/EBPα protein is regulated by post-translational phosphorylation and sumoylation. For example, FLT3 tyrosine kinase inhibitors and extra-cellular signal-regulated kinases 1 and/or 2 (ERK1/2) block serine-21 phosphorylation of C/EBPα, which increases the granulocytic differentiation potential of the C/EBPα protein [Radomska et al., Journal of Experimental Medicine, vol. 203(2), 371-381 (2006) and Ross et al., Molecular and Cellular Biology, vol. 24(2), 675-686 (2004)]. In addition, C/EBPα translation can be efficiently induced by 2-cyano-3,12-dioxoolean-1,9-dien-28-oic acid (CDDO), which alters the ratio of the C/EBPα protein isoforms in favor of the full-length p42 form over p30 form thereby inducing granulocytic differentiation [Koschmieder et al., Blood, vol. 110(10), 3695-3705 (2007)].

The C/EBPα gene is an intronless gene located on chromosome 19q13.1. Most eukaryotic cells use RNA-complementarity as a mechanism for regulating gene expression. One example is the RNA interference (RNAi) pathway which uses double stranded short interfering RNAs to knockdown gene expression via the RNA-induced silencing complex (RISC). It is now established that short duplex RNA oligonucleotides also have the ability to target the promoter regions of genes and mediate transcriptional activation of these genes and they have been referred to as RNA activation (RNAa), antigene RNA (agRNA) or short activating RNA (saRNA) [Li et al., PNAS, vol. 103, 17337-17342 (2006)]. saRNA induced activation of genes is conserved in other mammalian species including mouse, rat, and non-human primates and is fast becoming a popular method for studying the effects of endogenous up-regulation of genes.

Thus, there is a need for targeted modulation of C/EBPα for therapeutic purposes with saRNA.

### SUMMARY OF THE INVENTION

One aspect of the invention provides a method of increasing white blood cell (WBC) or neutrophils (NEU) levels comprising administering a synthetic isolated saRNA which up-regulates expression of C/EBPα gene.

Another aspect of the invention provides a method of reducing inflammation of a cell comprising administering a synthetic isolated saRNA which up-regulates the expression of C/EBPα gene.

Yet another aspect of the invention provides dosing regimens of a synthetic isolated saRNA which up-regulates the expression of C/EBPα gene.

The details of various embodiments of the invention are set forth in the description below. Other features, objects, and advantages of the invention will be apparent from the description and the drawings, and from the claims.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead being placed upon illustrating the principles of various embodiments of the invention.

FIG. 1 is a schematic illustrating the relationships among the nucleic acid moieties involved in the function of an saRNA of the invention.

FIG. 2 shows the white blood cell (WBC) levels after treatment with MTL-CEBPA.

FIG. 3 shows the neutrophil (NEU) levels after treatment with MTL-CEBPA.

FIG. 4 shows CEBPA-mRNA levels in white blood cells after treatment with MTL-CEBPA.

FIG. 5A-5C show inflammation marker levels in white blood cells after treatment with MTL-CEBPA. FIG. 5A: IFN-gamma; FIG. 5B: NFkB; FIG. 5C: IL6-R.

FIG. 6A-6D show downstream CEBPA target levels in white blood cells after treatment with MTL-CEBPA. FIG. 6A: CSF1R; FIG. 6B: CSF3R; FIG. 6C: ELANE; FIG. 6D: SPI1.

FIG. 7A shows the impact of MTL-CEBPA treatments on tumor volumes in the DEN study discussed in Example 4. FIG. 7B shows the impact of MTL-CEBPA treatments on albumin levels. FIG. 7C shows the impact of MTL-CEBPA treatments on bilirubin levels.

### DETAILED DESCRIPTION OF THE INVENTION

The present invention provides compositions, methods and kits for modulating C/EBPα gene expression and/or function for therapeutic purposes. These compositions, methods and kits comprise nucleic acid constructs that target a C/EBPα transcript.

C/EBPα protein is known as a critical regulator of metabolic processes and cell proliferation. Modulating C/EBPα gene has great potentials for therapeutic purposes. The present invention addresses this need by providing nucleic acid constructs targeting a C/EBPα transcript, wherein the nucleic acid constructs may include single or double stranded DNA or RNA with or without modifications.

C/EBPα gene as used herein is a double-stranded DNA comprising a coding strand and a template strand. It may also be referred to the target gene in the present application.

The terms “C/EBPα transcript”, “C/EBPα target transcript” or “target transcript” in the context may be C/EBPα mRNA encoding C/EBPα protein. C/EBPα mRNA is transcribed from the template strand of C/EBPα gene and may exist in the mitochondria.

The antisense RNA of the C/EBPα gene transcribed from the coding strand of the C/EBPα gene is called a target antisense RNA transcript herein after. The target antisense RNA transcript may be a long non-coding antisense RNA transcript.

The terms “small activating RNA”, “short activating RNA”, or “saRNA” in the context of the present invention means a single-stranded or double-stranded RNA that upregulates or has a positive effect on the expression of a specific gene. The saRNA may be single-stranded of 14 to 30 nucleotides. The saRNA may also be double-stranded, each strand comprising 14 to 30 nucleotides. The gene is called the target gene of the saRNA. A saRNA that upregulates the expression of the C/EBPα gene is called a “C/EBPα-saRNA” and the C/EBPα gene is the target gene of the C/EBPα-saRNA.

In one embodiment, C/EBPα-saRNA targeting a C/EBPα target antisense RNA transcript upregulates C/EBPα gene expression and/or function.

The terms “target” or “targeting” in the context mean having an effect on a C/EBPα gene. The effect may be direct or indirect. Direct effect may be caused by complete or partial hybridization with the C/EBPα target antisense RNA transcript. Indirect effect may be upstream or downstream.

C/EBPα-saRNA may have a downstream effect on a biological process or activity. In such embodiments, C/EBPα-saRNA may have an effect (either upregulating or downregulating) on a second, non-target transcript.

The term “gene expression” in the context may include the transcription step of generating C/EBPα mRNA from C/EBPα gene or the translation step generating C/EBPα protein from C/EBPα mRNA. An increase of C/EBPα mRNA and an increase of C/EBPα protein both indicate an increase or a positive effect of C/EBPα gene expression.

By “upregulation” or “activation” of a gene is meant an increase in the level of expression of a gene, or levels of the polypeptide(s) encoded by a gene or the activity thereof, or levels of the RNA transcript(s) transcribed from the template strand of a gene above that observed in the absence of the saRNA of the present invention. The saRNA of the present invention may have a direct or indirect upregulating effect on the expression of the target gene.

In one embodiment, the saRNA of the present invention may show efficacy in proliferating cells. As used herein with respect to cells, “proliferating” means cells which are growing and/or reproducing rapidly.

I. Composition of the Invention

One aspect of the present invention provides pharmaceutical compositions comprising a saRNA that upregulates CEBPA gene, and at least one pharmaceutically acceptable carrier. Such a saRNA is referred herein after as “C/EBPα-saRNA”, or “saRNA of the present invention”, used interchangeably in this application.

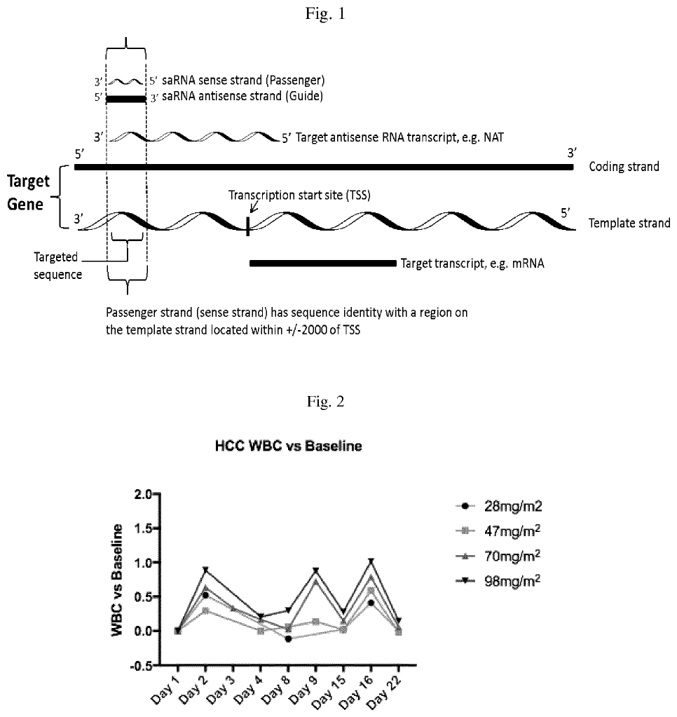

The C/EBPα-saRNA has 14-30 nucleotides and comprises a sequence that is at least 80%, 90%, 95%, 98%, 99% or 100% complementary to a targeted sequence on the template strand of the C/EBPα gene. The targeted sequence may have the same length, i.e., the same number of nucleotides, as the saRNA and/or the reverse complement of the saRNA. The relationships among the saRNAs, a target gene, a coding strand of the target gene, a template strand of the target gene, a targeted sequence/target site, and the TSS are shown in FIG. 1.

In some embodiments, the targeted sequence comprises at least 14 and less than 30 nucleotides.

In some embodiments, the targeted sequence has 19, 20, 21, 22, or 23 nucleotides.

In some embodiments, the location of the targeted sequence is situated within a promoter area of the template strand.

In some embodiments, the targeted sequence of the C/EBPα-saRNA is located within a TSS (transcription start site) core of the template stand of the C/EBPα gene. A “TSS core” or “TSS core sequence” as used herein, refers to a region between 2000 nucleotides upstream and 2000 nucleotides downstream of the TSS (transcription start site). Therefore, the TSS core comprises 4001 nucleotides and the TSS is located at position 2001 from the 5′ end of the TSS core sequence. CEBPA TSS core sequence is show in the table below:

| CEBPA mRNA | CEBPA protein | CEBPA TSS core | CEBPA TSS core |
|---|---|---|---|
| REF. No. | REF. No. | genomic location | sequence ID No. |
| NM_001285829 | NP_001272758 | chr19:33302564 | SEQ ID No. 3 |
| NM_001287424 | NP_001274353 | minus strand | |
| NM_001287435 | NP_001274364 | | |
| NM_004364 | NP_004355 | | |

In some embodiments, the targeted sequence is located between 1000 nucleotides upstream and 1000 nucleotides downstream of the TSS.

In some embodiments, the targeted sequence is located between 500 nucleotides upstream and 500 nucleotides downstream of the TSS.

In some embodiments, the targeted sequence is located between 250 nucleotides upstream and 250 nucleotides downstream of the TSS.

In some embodiments, the targeted sequence is located between 100 nucleotides upstream and 100 nucleotides downstream of the TSS.

In some embodiments, the targeted sequence is located upstream of the TSS in the TSS core. The targeted sequence may be less than 2000, less than 1000, less than 500, less than 250, or less than 100 nucleotides upstream of the TSS.

In some embodiments, the targeted sequence is located downstream of the TSS in the TSS core. The targeted sequence may be less than 2000, less than 1000, less than 500, less than 250, or less than 100 nucleotides downstream of the TSS.

In some embodiments, the targeted sequence is located +/−50 nucleotides surrounding the TSS of the TSS core. In some embodiments, the targeted sequence substantially overlaps the TSS of the TSS core. In some embodiments, the targeted sequence begins or ends at the TSS of the TSS core. In some embodiments, the targeted sequence overlaps the TSS of the TSS core by 1, 2, 3, 4, 5, 6, 7, 8, 9, 10, 11, 12, 13, 14, 15, 16, 17, 18 or 19 nucleotides in either the upstream or downstream direction.

The location of the targeted sequence on the template strand is defined by the location of the 5′ end of the targeted sequence. The 5′ end of the targeted sequence may be at any position of the TSS core and the targeted sequence may start at any position selected from position 1 to position 4001 of the TSS core. For reference herein, when the 5′ most end of the targeted sequence from position 1 to position 2000 of the TSS core, the targeted sequence is considered upstream of the TSS and when the 5′ most end of the targeted sequence is from position 2002 to 4001, the targeted sequence is considered downstream of the TSS. When the 5′ most end of the targeted sequence is at nucleotide 2001, the targeted sequence is considered to be a TSS centric sequence and is neither upstream nor downstream of the TSS.

For further reference, for example, when the 5′ end of the targeted sequence is at position 1600 of the TSS core, i.e., it is the 1600^{th} nucleotide of the TSS core, the targeted sequence starts at position 1600 of the TSS core and is considered to be upstream of the TSS.

In one embodiment, the saRNA of the present invention may have two strands that form a duplex, one strand being a guide strand. The saRNA duplex is also called a double-stranded saRNA. A double-stranded saRNA or saRNA duplex, as used herein, is a saRNA that includes more than one, and preferably, two, strands in which interstrand hybridization can form a region of duplex structure. The two strands of a double-stranded saRNA are referred to as an antisense strand or a guide strand, and a sense strand or a passenger strand.

In some embodiments, the C/EBPα-saRNA may comprising any C/EBPα-saRNA disclosed in WO2015/075557 or WO2016/170349 to MiNA Therapeutics Limited, the contents of each of which are incorporated herein by reference in their entirety, such as saRNAs in Table 1, Table 1A, Table 3-1 and Table 3-2, AW51, and CEBPA-51 disclosed in WO2016/170349.

In some embodiments, the C/EBPα-saRNA may be modified and may comprising any modification disclosed in WO2016/170349 to MiNA Therapeutics Limited.

In one embodiment, the C/EBPα-saRNA is CEBPA-51 (or CEBPA51), which is an saRNA duplex that upregulates C/EBPα. Its design, sequences, and compositions/formulations are disclosed in the Detailed Description and Examples of WO2016/170349 to MiNA Therapeutics Limited. The sequences of the sense and antisense strands of CEBPA-51 are shown in Table 1.

TABLE 1
CEBPA-51 (CEBPA51) Sequences

| | | |
|---|---|---|
| Antisense | GACCAGUGACAAUGACCGCmUmU | SEQ ID No. 1 |
| Sense | (invabasic)mGmCGmGUCAUUmGUCAmCUGGUCmUmU | SEQ ID No. 2 |

mU, mG, and mC mean 2′-O-methyl modified U, G, and C.

invabasic=inverted abasic sugar cap.

The alignment of the strands is shown in the Table 2.

TABLE 2
CEBPA-51 Alignment of Strands

| | saRNA name | | | | | | |
|---|---|---|---|---|---|---|---|
| | CEBPA-51 Total base: 21 mer, including base modifications | | | | | | |
| mer | 3 | 6 | 9 | 12 | 15 | 18 | 21 |
| Sense strand | bmGmCG | mGUC | AUU | mGUC | AmCU | GGU | CmUmU |
| 5′ → 3′ | | | | | | | |
| (SEQ ID No. 2) | | | | | | | |
| Complementary | mUmUC | GCC | AGU | AAC | AGU | GAC | CAG |
| antisense strand | | | | | | | |
| 3′ → 5′ | | | | | | | |
| (SEQ ID No. 1) | | | | | | | |

Definition of symbols: A, U, G, C are 2′-OH ribonucleotides, mU, mG, mC are 2′-O-methyl ribonucleotides, b = inverted abasic sugar cap.

CEBPA-51 is encapsulated into liposomes (NOV340 SMARTICLES® technology owned by Marina Biotech) to make MTL-CEBPA. The lipid components of the NOV340 SMARTICLES® are comprised of 1-palmitoyl-2-oleoyl-sn-glycero-3-phosphocholine (POPC), 1,2-dioleoyl-sn-glycero-3-phosphoethanolamine (DOPE), cholesteryl-hemisuccinate (CHEMS), and 4-(2-aminoethyl)-morpholino-cholesterolhemisuccinate (MOCHOL). NOV340 SMARTICLES® consists of POPC, DOPE, CHEMS and MOCHOL in the molar ratio of 6:24:23:47. These nanoparticles are anionic at physiological pH, and their specific lipid ratio imparts a “pH-tunable” character and a charge to the liposomes, which changes depending upon the surrounding pH of the microenvironment to facilitate movement across physiologic membranes. SMARTICLES® nanoparticles are sized to avoid extensive immediate hepatic sequestration, with an average diameter of approximately about 50-about 150 nm, or about 100-about 120 nm, facilitating more prolonged systemic distribution and improved serum stability after i.v. injection leading to broader tissue distribution with high levels in liver, spleen and bone marrow reported.

MTL-CEBPA also comprises the buffer forming excipients such as sucrose and phosphate-salts. Qualitative and quantitative composition of MTL-CEBPA (2.5 mg/ml) are shown in Table 3.

TABLE 3
MTL-CEBPA Composition

| | | | Quantity | |
|---|---|---|---|---|
| Name of Ingredient | Function | Reference | (per ml) | |
| CEBPA-51 (saRNA) | Active pharmaceutical | Manufacturer's | 2.5 | mg/ml |
| | ingredient | specifications | | |
| 1-palmitoyl-2-oleoyl-sn-glycero-3- | Membrane forming lipid | Manufacturer's | 4.65 | mg/ml |
| phosphocholine (POPC) | | specifications | | |
| 1,2-dioleoyl-sn-glycero-3- | Membrane forming | Manufacturer's | 18.0 | mg/ml |
| phosphoethanolamine (DOPE) | fusogenic lipid | specifications | | |
| Cholesteryl hemisuccinate (CHEMS) | Anionic ampotheric lipid | Manufacturer's | 11.3 | mg/ml |
| | | specifications | | |
| Cholesteryl-4-[[2-(4- | Cationic amphoteric lipid | Manufacturer's | 27.0 | mg/ml |
| morpholinyl)ethyl]amino]-4-oxobutanoate | | specifications | | |
| (MOCHOL) | | | | |
| Sucrose | Cryoprotectant, | BP, JP, NF, EP | 92.4 | mg/ml |
| | osmolality control | | | |
| Disodium hydrogen phosphate, dihydrate | Buffer pH adjustment | BP, USP, EP | 1.44 | mg/ml |
| Potassium dihydrogen phosphate | Buffer pH adjustment | EP, BP, NF | 0.2 | mg/ml |
| Potassium chloride (KCl) | Ionic strength adjuster | EP, BP, USP | 0.2 | mg/ml |
| Water for injection (WFI) | Solvent | WFI (USP, EP) | qs 1 | ml |

II. Methods of Use

One aspect of the present invention provides methods of using C/EBPα-saRNA. As disclosed in WO2016/170349 to MiNA Therapeutics Limited, C/EBPα-saRNA may be used to regulate metabolics and/or treat hyperproliferation disorders. The present disclosure provides additional uses of C/EBPα-saRNAs or compositions thereof.

### White Blood Cells and Neutrophils

White blood cells (WBCs), also called leukocytes or leucocytes, are the cells of the immune system that are involved in protecting the body against both infectious disease and foreign invaders. Neutrophils are the most abundant type of white blood cells in human. They are formed from stem cells in bone marrow and are an essential part of the innate immune system.

Some cancers, including blood and bone marrow cancers, and cancer treatments may cause low WBC count and/or low neutrophil count. For example, certain chemotherapy drugs can damage the bone marrow of the patient. Patients receiving radiation therapy to bones that contain the bone marrow may also experience low levels of WBCs and neutrophils. Low WBC count and/or low neutrophil level can lead to a higher risk of infection and other complications. Low WBC count and/or low neutrophil level may also delay cancer treatments.

In some embodiments, C/EBPα-saRNAs or C/EBPα-saRNA compositions, such as CEBPA-51 and/or MTL-CEBPA, are used to increase CEBPA mRNA and/or CEBPA protein levels in bone marrow hematopoietic stem and progenitor cells.

In some embodiments, C/EBPα-saRNAs or C/EBPα-saRNA compositions, such as CEBPA-51 and/or MTL-CEBPA, are used to increase white blood cells and/or neutrophils of a patient. Not willing to be bound by any theory, CEBPA mRNA and/or CEBPA protein levels in bone marrow hematopoietic stem and progenitor cells are increased, which drives bone marrow hematopoietic stem and progenitor cells preferentially down the myeloid lineage leading to the increase in neutrophils. In some embodiments, WBC count and/or neutrophil count may increase at least 10%, 20%, 30%, 40%, 50%, 60%, 70%, 80%, 90%, 100%, 110%, 120%, 130%, 140%, 150%, two folds, three folds, four folds, five folds, or ten folds compared to the WBC count and/or neutrophil count before CEBPA-saRNA administration. The increase may occur around 30 min, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 30 hours, 36 hours, 42 hours, or 48 hours after CEBPA-saRNA administration. The increase may last at least 12 hours, 24 hours, 30 hours, 36 hours, 7 days, or 14 days

In some embodiments, the patient has cancer. Examples of cancer include, but not limited to, cervical cancer, uterine cancer, ovarian cancer, kidney cancer, gallbladder cancer, liver cancer, head and neck cancer, squamous cell carcinoma, gastrointestinal cancer, breast cancer, prostate cancer, testicular cancer, lung cancer, non-small cell lung cancer, non-Hodgkin's lymphoma, multiple myeloma, leukemia (such as acute lymphocytic leukemia, chronic lymphocytic leukemia, acute myelogenous leukemia, and chronic myelogenous leukemia), brain cancer (e.g. astrocytoma, glioblastoma, medulloblastoma), neuroblastoma, sarcomas, colon cancer, rectum cancer, stomach cancer, anal cancer, bladder cancer, endometrial cancer, plasmacytoma, lymphomas, retinoblastoma, Wilm's tumor, Ewing sarcoma, melanoma and other skin cancers. The liver cancer may include, but not limited to, cholangiocarcinoma, hepatoblastoma, haemangio sarcoma, or hepatocellular carcinoma (HCC). In some examples, the patient has a liver cancer (such as hepatocellular carcinoma (HCC)), a cancer of the blood and/or bone marrow (such as leukemia), or a cancer that metastasize to the bone marrow. In some embodiments, the patient is receiving or has received at least one treatment for cancer, such as chemotherapy drugs and/or radiation therapy.

In some embodiments, at least one hematopoietic cell growth factor is administered to the patient in addition to C/EBPα-saRNAs or C/EBPα-saRNA compositions, such as CEBPA-51 and/or MTL-CEBPA. Examples of cell growth factors include granulocyte colony-stimulating factor (G-CSF) and granulocyte-macrophage colony-stimulating factor (GM-CSF). CSFs include filgrastim (Neupogen, Zarxio), sargramostim (Leukine), and pegfilgrastim (Neulasta). The growth factors may be administered simultaneously, separately, or sequentially with to C/EBPα-saRNAs or C/EBPα-saRNA compositions.

In some embodiments, antibiotics such as Imipenem, Meropenem, Ceftazidime or Ciprofloxacin by way of examples or anti-fungal agents such as Voriconazole or Caspofungin by way of examples may be administered in combination with C/EBPα-saRNAs or C/EBPα-saRNA compositions, such as CEBPA-51 and/or MTL-CEBPA.

### Inflammation

In some embodiments, C/EBPα-saRNAs or C/EBPα-saRNA compositions, such as CEBPA-51 and/or MTL-CEBPA, are used to reduce inflammation of a patient. This may be demonstrated by the decreased inflammation biomarker levels.

In some embodiments, C/EBPα-saRNAs or C/EBPα-saRNA compositions, such as CEBPA-51 and/or MTL-CEBPA, are used to reduce the level of interferon gamma (IFN-gamma) in a cell. The cell may be a white blood cell (WBC). IFN-gamma level may decrease at least 10%, 20%, 30%, 40%, 50%, 60%, 70%, or 80% compared to the level before CEBPA-saRNA administration.

In some embodiments, C/EBPα-saRNAs or C/EBPα-saRNA compositions, such as CEBPA-51 and/or MTL-CEBPA, are used to reduce the level of nuclear factor kappa-light-chain-enhancer of activated B cells (NFkB) in a cell. The cell may be a WBC. NFkB level may decrease at least 10%, 20%, 30%, 40%, 50%, 60%, 70%, or 80% compared to the level before CEBPA-saRNA administration.

In some embodiments, C/EBPα-saRNAs or C/EBPα-saRNA compositions, such as CEBPA-51 and/or MTL-CEBPA, are used to reduce the level of interleukin 6 receptor (IL6-R) in a cell. The cell may be a WBC. IL6-R level may decrease at least 10%, 20%, 30%, 40%, 50%, 60%, 70%, or 80% compared to the level before CEBPA-saRNA administration.

In some embodiments other anti-inflammatory drugs such Non-steroidal anti-inflammatory drugs (NSAIDs) such as Ibuprofen or TNFalpha inhibitors such as Adalimumab, Etanercept or other disease-modifying anti-rheumatic drugs (DMARDs) such as methotrexate may be administered in combination with C/EBPα-saRNAs or C/EBPα-saRNA compositions, such as CEBPA-51 and/or MTL-CEBPA.

### Dosing

In some embodiments, C/EBPα-saRNAs or C/EBPα-saRNA compositions, such as CEBPA-51 and/or MTL-CEBPA, are administered once every day, once every 2 days, once every 3 days, once every 4 days, or once every 5 days.

In some embodiments, at least two doses of C/EBPα-saRNAs or C/EBPα-saRNA compositions, such as CEBPA-51 and/or MTL-CEBPA, are administered to a subject. The subject may have a liver disease, such as liver cancer, non-alcoholic steatohepatitis (NASH), steatosis, liver damage, liver failure, or liver fibrosis. The doses are less than 7 days apart. In one embodiment, CEBPA-51 and/or MTL-CEBPA is administered every 24 hours. In one embodiment, CEBPA-51 and/or MTL-CEBPA is administered every 48 hours. In some embodiments, the subject receives 2 doses of C/EBPα-saRNAs or C/EBPα-saRNA compositions, wherein the doses are 24 hours or 48 hours apart. In some embodiments, the subject receives 3 doses of C/EBPα-saRNAs or C/EBPα-saRNA compositions, wherein the doses are 24 hours or 48 hours apart.

In some embodiments, the patient receives at least 2 doses, e.g, 3 doses, 4 doses, 5 doses, 6 doses, 7 doses, 8 doses, 9 doses, or 10 doses, of C/EBPα-saRNAs or C/EBPα-saRNA compositions, such as CEBPA-51 and/or MTL-CEBPA.

In some embodiments, C/EBPα-saRNAs or C/EBPα-saRNA compositions, such as CEBPA-51 and/or MTL-CEBPA, are administered for a period of at least 2 days, such as 3 days, 4 days, 5 days, 6 days, 1 week, 8 days, 9 days, 10 days, 2 weeks, 3 weeks, 4 weeks, 5 weeks, 6 weeks, 2 months, 3 months, 4 months, 5 months, 6 months, 7 months, 8 months, 9 months, 10 months, 11 months, or a year.

In one embodiment, CEBPA-51 and/or MTL-CEBPA is administered every 24 hours for a period of at least 2 days, such as 3 days, 4 days, 5 days, 6 days, 1 week, 8 days, 9 days, 10 days, 2 weeks, 3 weeks, 4 weeks, 5 weeks, 6 weeks, 2 months, 3 months, 4 months, 5 months, 6 months, 7 months, 8 months, 9 months, 10 months, 11 months, or a year.

In one embodiment, CEBPA-51 and/or MTL-CEBPA is administered every 48 hours for a period of at least 2 days, such as 3 days, 4 days, 5 days, 6 days, 1 week, 8 days, 9 days, 10 days, 2 weeks, 3 weeks, 4 weeks, 5 weeks, 6 weeks, 2 months, 3 months, 4 months, 5 months, 6 months, 7 months, 8 months, 9 months, 10 months, 11 months, or a year.

In some embodiments, C/EBPα-saRNAs or C/EBPα-saRNA compositions, such as CEBPA-51 and/or MTL-CEBPA, are administered via intravenous infusion over 60 minutes. Doses are between about 20 to about 200 mg/m², such as about 30 mg/m², about 50 mg/m², about 80 mg/m², about 100 mg/m², about 120 mg/m², about 150 mg/m², about 160 mg/m², or about 180 mg/m².

The dosing regimen disclosed in the present application may apply to any indication or disorder that can be treated with C/EBPα-saRNAs or C/EBPα-saRNA compositions, such as regulating white blood cells and neutrophils and reducing inflammation as discussed in the present application, and any use disclosed in WO2016/170349 to MiNA Therapeutics Limited, including treating metabolics disorders and/or hyperproliferation disorders.

III. Kits and Devices

### Kits

The invention provides a variety of kits for conveniently and/or effectively carrying out methods of the present invention. Typically, kits will comprise sufficient amounts and/or numbers of components to allow a user to perform multiple treatments of a subject(s) and/or to perform multiple experiments.

In one embodiment, the present invention provides kits for regulate the expression of genes in vitro or in vivo, comprising CEBPA-51 or a combination of CEBPA-51, saRNA modulating other genes, siRNAs, miRNAs, and/or other active agents. The kit may further comprise packaging and instructions and/or a delivery agent to form a formulation composition. The delivery agent may comprise a saline, a buffered solution, a lipidoid, a dendrimer or any delivery agent disclosed herein. Non-limiting examples of genes include C/EBPα, other members of C/EBP family, albumin gene, alphafectoprotein gene, liver specific factor genes, growth factors, nuclear factor genes, tumor suppressing genes, pluripotency factor genes.

In another embodiment, the present invention provides kits to regulate the proliferation of cells, comprising CEBPA-51, provided in an amount effective to regulate white blood cells and neutrophils and/or to reduce inflammation; optionally other active agent to further regulate white blood cells and neutrophils and/or to reduce inflammation; and packaging and instructions and/or a delivery agent to form a formulation composition.

In another embodiment, the present invention provides kits for regulating white blood cells and neutrophils and/or for reducing inflammation, comprising CEBPA-51; optionally other active agent to regulate white blood cells and neutrophils and/or to reduce inflammation; and packaging and instructions and/or a delivery agent to form a formulation composition.

In another embodiment, the present invention provides kits for regulating white blood cells and neutrophils and/or for reducing inflammation, comprising CEBPA-51; optionally siRNAs, eRNAs and lncRNAs; and packaging and instructions and/or a delivery agent to form a formulation composition.

### Devices

The present invention provides for devices which may incorporate CEBPA-51. These devices contain in a stable formulation available to be immediately delivered to a subject in need thereof, such as a human patient. Non-limiting examples of such a subject include a subject with hyperproliferative disorders such as cancer, tumor, or liver cirrhosis; and metabolics disorders such as NAFLD, obesity, high LDL cholesterol, or type II diabetes.

Non-limiting examples of the devices include a pump, a catheter, a needle, a transdermal patch, a pressurized olfactory delivery device, iontophoresis devices, multi-layered microfluidic devices. The devices may be employed to deliver CEBPA-51 according to single, multi- or split-dosing regiments. The devices may be employed to deliver CEBPA-51 across biological tissue, intradermal, subcutaneously, or intramuscularly. More examples of devices suitable for delivering oligonucleotides are disclosed in International Publication WO 2013/090648 filed Dec. 14, 2012, the contents of which are incorporated herein by reference in their entirety.

### Definitions

For convenience, the meaning of certain terms and phrases used in the specification, examples, and appended claims, are provided below. If there is an apparent discrepancy between the usage of a term in other parts of this specification and its definition provided in this section, the definition in this section shall prevail.

About: As used herein, the term “about” means+/−10% of the recited value.

Administered in combination: As used herein, the term “administered in combination” or “combined administration” means that two or more agents, e.g., saRNA, are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently close together such that a combinatorial (e.g., a synergistic) effect is achieved.

Amino acid: As used herein, the terms “amino acid” and “amino acids” refer to all naturally occurring L-alpha-amino acids. The amino acids are identified by either the one-letter or three-letter designations as follows: aspartic acid (Asp: D), isoleucine (Ile: I), threonine (Thr: T), leucine (Leu: L), serine (Ser: S), tyrosine (Tyr: Y), glutamic acid (Glu: E), phenylalanine (Phe: F), proline (Pro: P), histidine (His: H), glycine (Gly: G), lysine (Lys: K), alanine (Ala: A), arginine (Arg: R), cysteine (Cys: C), tryptophan (Trp: W), valine (Val: V), glutamine (Gln: Q) methionine (Met: M), asparagines (Asn: N), where the amino acid is listed first followed parenthetically by the three and one letter codes, respectively.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Bifunctional: As used herein, the term “bifunctional” refers to any substance, molecule or moiety which is capable of or maintains at least two functions. The functions may affect the same outcome or a different outcome. The structure that produces the function may be the same or different.

Biocompatible: As used herein, the term “biocompatible” means compatible with living cells, tissues, organs or systems posing little to no risk of injury, toxicity or rejection by the immune system.

Biodegradable: As used herein, the term “biodegradable” means capable of being broken down into innocuous products by the action of living things.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, the saRNA of the present invention may be considered biologically active if even a portion of the saRNA is biologically active or mimics an activity considered biologically relevant.

Cancer: As used herein, the term “cancer” in an individual refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an individual, or may circulate in the blood stream as independent cells, such as leukemic cells.

Cell growth: As used herein, the term “cell growth” is principally associated with growth in cell numbers, which occurs by means of cell reproduction (i.e. proliferation) when the rate of the latter is greater than the rate of cell death (e.g. by apoptosis or necrosis), to produce an increase in the size of a population of cells, although a small component of that growth may in certain circumstances be due also to an increase in cell size or cytoplasmic volume of individual cells. An agent that inhibits cell growth can thus do so by either inhibiting proliferation or stimulating cell death, or both, such that the equilibrium between these two opposing processes is altered.

Cell type: As used herein, the term “cell type” refers to a cell from a given source (e.g., a tissue, organ) or a cell in a given state of differentiation, or a cell associated with a given pathology or genetic makeup.

Chromosome: As used herein, the term “chromosome” refers to an organized structure of DNA and protein found in cells.

Complementary: As used herein, the term “complementary” as it relates to nucleic acids refers to hybridization or base pairing between nucleotides or nucleic acids, such as, for example, between the two strands of a double-stranded DNA molecule or between an oligonucleotide probe and a target are complementary.

Condition: As used herein, the term “condition” refers to the status of any cell, organ, organ system or organism. Conditions may reflect a disease state or simply the physiologic presentation or situation of an entity. Conditions may be characterized as phenotypic conditions such as the macroscopic presentation of a disease or genotypic conditions such as the underlying gene or protein expression profiles associated with the condition. Conditions may be benign or malignant.

Controlled Release: As used herein, the term “controlled release” refers to a pharmaceutical composition or compound release profile that conforms to a particular pattern of release to effect a therapeutic outcome.

Cytostatic: As used herein, “cytostatic” refers to inhibiting, reducing, suppressing the growth, division, or multiplication of a cell (e.g., a mammalian cell (e.g., a human cell)), bacterium, virus, fungus, protozoan, parasite, prion, or a combination thereof.

Cytotoxic: As used herein, “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

Delivery: As used herein, “delivery” refers to the act or manner of delivering a compound, substance, entity, moiety, cargo or payload.

Delivery Agent: As used herein, “delivery agent” refers to any substance which facilitates, at least in part, the in vivo delivery of a saRNA of the present invention to targeted cells.

Destabilized: As used herein, the term “destable,” “destabilize,” or “destabilizing region” means a region or molecule that is less stable than a starting, wild-type or native form of the same region or molecule.

Detectable label: As used herein, “detectable label” refers to one or more markers, signals, or moieties which are attached, incorporated or associated with another entity that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the peptides, proteins or polynucleotides, e.g, saRNA, disclosed herein. They may be within the amino acids, the peptides, proteins, or polynucleotides located at the N- or C-termini or 5′ or 3′ termini as the case may be.

Encapsulate: As used herein, the term “encapsulate” means to enclose, surround or encase.

Engineered: As used herein, embodiments of the invention are “engineered” when they are designed to have a feature or property, whether structural or chemical, that varies from a starting point, wild type or native molecule.

Equivalent subject: As used herein, “equivalent subject” may be e.g. a subject of similar age, sex and health such as liver health or cancer stage, or the same subject prior to treatment according to the invention. The equivalent subject is “untreated” in that he does not receive treatment with a saRNA according to the invention. However, he may receive a conventional anti-cancer treatment, provided that the subject who is treated with the saRNA of the invention receives the same or equivalent conventional anti-cancer treatment.

Exosome: As used herein, “exosome” is a vesicle secreted by mammalian cells.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Feature: As used herein, a “feature” refers to a characteristic, a property, or a distinctive element.

Formulation: As used herein, a “formulation” includes at least a saRNA of the present invention and a delivery agent.

Fragment: A “fragment,” as used herein, refers to a portion. For example, fragments of proteins may comprise polypeptides obtained by digesting full-length protein isolated from cultured cells.

Functional: As used herein, a “functional” biological molecule is a biological molecule in a form in which it exhibits a property and/or activity by which it is characterized.

Gene: As used herein, the term “gene” refers to a nucleic acid sequence that comprises control and most often coding sequences necessary for producing a polypeptide or precursor. Genes, however, may not be translated and instead code for regulatory or structural RNA molecules.

A gene may be derived in whole or in part from any source known to the art, including a plant, a fungus, an animal, a bacterial genome or episome, eukaryotic, nuclear or plasmid DNA, cDNA, viral DNA, or chemically synthesized DNA. A gene may contain one or more modifications in either the coding or the untranslated regions that could affect the biological activity or the chemical structure of the expression product, the rate of expression, or the manner of expression control. Such modifications include, but are not limited to, mutations, insertions, deletions, and substitutions of one or more nucleotides. The gene may constitute an uninterrupted coding sequence or it may include one or more introns, bound by the appropriate splice junctions.

Gene expression: As used herein, the term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

Genome: The term “genome” is intended to include the entire DNA complement of an organism, including the nuclear DNA component, chromosomal or extrachromosomal DNA, as well as the cytoplasmic domain (e.g., mitochondrial DNA).

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the invention, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the invention, two protein sequences are considered to be homologous if the proteins are at least about 50%, 60%, 70%, 80%, or 90% identical for at least one stretch of at least about 20 amino acids.

The term “hyperproliferative cell” may refer to any cell that is proliferating at a rate that is abnormally high in comparison to the proliferating rate of an equivalent healthy cell (which may be referred to as a “control”). An “equivalent healthy” cell is the normal, healthy counterpart of a cell. Thus, it is a cell of the same type, e.g. from the same organ, which performs the same functions(s) as the comparator cell. For example, proliferation of a hyperproliferative hepatocyte should be assessed by reference to a healthy hepatocyte, whereas proliferation of a hyperproliferative prostate cell should be assessed by reference to a healthy prostate cell.

By an “abnormally high” rate of proliferation, it is meant that the rate of proliferation of the hyperproliferative cells is increased by at least 20, 30, 40%, or at least 45, 50, 55, 60, 65, 70, 75%, or at least 80%, as compared to the proliferative rate of equivalent, healthy (non-hyperproliferative) cells. The “abnormally high” rate of proliferation may also refer to a rate that is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, compared to the proliferative rate of equivalent, healthy cells.

The term “hyperproliferative cell” as used herein does not refer to a cell which naturally proliferates at a higher rate as compared to most cells, but is a healthy cell. Examples of cells that are known to divide constantly throughout life are skin cells, cells of the gastrointestinal tract, blood cells and bone marrow cells. However, when such cells proliferate at a higher rate than their healthy counterparts, then they are hyperproliferative.

Hyperproliferative disorder: As used herein, a “hyperproliferative disorder” may be any disorder which involves hyperproliferative cells as defined above. Examples of hyperproliferative disorders include neoplastic disorders such as cancer, psoriatic arthritis, rheumatoid arthritis, gastric hyperproliferative disorders such as inflammatory bowel disease, skin disorders including psoriasis, Reiter's syndrome, Pityriasis rubra pilaris, and hyperproliferative variants of the disorders of keratinization.

The skilled person is fully aware of how to identify a hyperproliferative cell. The presence of hyperproliferative cells within an animal may be identifiable using scans such as X-rays, MRI or CT scans. The hyperproliferative cell may also be identified, or the proliferation of cells may be assayed, through the culturing of a sample in vitro using cell proliferation assays, such as MTT, XTT, MTS or WST-1 assays. Cell proliferation in vitro can also be determined using flow cytometry.

Identity: As used herein, the term “identity” refers to the overall relatedness between polymeric molecules, e.g., between oligonucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically a reduction in the level of an mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

Label: The term “label” refers to a substance or a compound which is incorporated into an object so that the substance, compound or object may be detectable.

Linker: As used herein, a linker refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker may be of sufficient length as to not interfere with incorporation into a nucleic acid sequence. The linker can be used for any useful purpose, such as to form saRNA conjugates, as well as to administer a payload, as described herein. Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers and derivatives thereof. Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond can be cleaved for example by the use of tris(2-carboxyethyl)phosphine (TCEP), or other reducing agents, and/or photolysis, as well as an ester bond can be cleaved for example by acidic or basic hydrolysis.

Metastasis: As used herein, the term “metastasis” means the process by which cancer spreads from the place at which it first arose as a primary tumor to distant locations in the body. Metastasis also refers to cancers resulting from the spread of the primary tumor. For example, someone with breast cancer may show metastases in their lymph system, liver, bones or lungs.

Modified: As used herein “modified” refers to a changed state or structure of a molecule of the invention. Molecules may be modified in many ways including chemically, structurally, and functionally. In one embodiment, the saRNA molecules of the present invention are modified by the introduction of non-natural nucleosides and/or nucleotides.

Naturally occurring: As used herein, “naturally occurring” means existing in nature without artificial aid.

Nucleic acid: The term “nucleic acid” as used herein, refers to a molecule comprised of one or more nucleotides, i.e., ribonucleotides, deoxyribonucleotides, or both. The term includes monomers and polymers of ribonucleotides and deoxyribonucleotides, with the ribonucleotides and/or deoxyribonucleotides being bound together, in the case of the polymers, via 5′ to 3′ linkages. The ribonucleotide and deoxyribonucleotide polymers may be single or double-stranded. However, linkages may include any of the linkages known in the art including, for example, nucleic acids comprising 5′ to 3′ linkages. The nucleotides may be naturally occurring or may be synthetically produced analogs that are capable of forming base-pair relationships with naturally occurring base pairs. Examples of non-naturally occurring bases that are capable of forming base-pairing relationships include, but are not limited to, aza and deaza pyrimidine analogs, aza and deaza purine analogs, and other heterocyclic base analogs, wherein one or more of the carbon and nitrogen atoms of the pyrimidine rings have been substituted by heteroatoms, e.g., oxygen, sulfur, selenium, phosphorus, and the like.

Patient: As used herein, “patient” refers to a subject who may seek or be in need of treatment, requires treatment, is receiving treatment, will receive treatment, or a subject who is under care by a trained professional for a particular disease or condition.

Peptide: As used herein, “peptide” is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Pharmaceutically acceptable solvate: The term “pharmaceutically acceptable solvate,” as used herein, means a compound of the invention wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmacologic effect: As used herein, a “pharmacologic effect” is a measurable biologic phenomenon in an organism or system which occurs after the organism or system has been contacted with or exposed to an exogenous agent. Pharmacologic effects may result in therapeutically effective outcomes such as the treatment, improvement of one or more symptoms, diagnosis, prevention, and delay of onset of disease, disorder, condition or infection. Measurement of such biologic phenomena may be quantitative, qualitative or relative to another biologic phenomenon. Quantitative measurements may be statistically significant. Qualitative measurements may be by degree or kind and may be at least 10%, 20%, 30%, 40%, 50%, 60%, 70%, 80%, 90% or more different. They may be observable as present or absent, better or worse, greater or less. Exogenous agents, when referring to pharmacologic effects are those agents which are, in whole or in part, foreign to the organism or system. For example, modifications to a wild type biomolecule, whether structural or chemical, would produce an exogenous agent. Likewise, incorporation or combination of a wild type molecule into or with a compound, molecule or substance not found naturally in the organism or system would also produce an exogenous agent. The saRNA of the present invention, comprises exogenous agents. Examples of pharmacologic effects include, but are not limited to, alteration in cell count such as an increase or decrease in neutrophils, reticulocytes, granulocytes, erythrocytes (red blood cells), megakaryocytes, platelets, monocytes, connective tissue macrophages, epidermal langerhans cells, osteoclasts, dendritic cells, microglial cells, neutrophils, eosinophils, basophils, mast cells, helper T cells, suppressor T cells, cytotoxic T cells, natural killer T cells, B cells, natural killer cells, or reticulocytes. Pharmacologic effects also include alterations in blood chemistry, pH, hemoglobin, hematocrit, changes in levels of enzymes such as, but not limited to, liver enzymes AST and ALT, changes in lipid profiles, electrolytes, metabolic markers, hormones or other marker or profile known to those of skill in the art.

Physicochemical: As used herein, “physicochemical” means of or relating to a physical and/or chemical property.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Prodrug: The present disclosure also includes prodrugs of the compounds described herein. As used herein, “prodrugs” refer to any substance, molecule or entity which is in a form predicate for that substance, molecule or entity to act as a therapeutic upon chemical or physical alteration. Prodrugs may by covalently bonded or sequestered in some way and which release or are converted into the active drug moiety prior to, upon or after administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

Prognosing: As used herein, the term “prognosing” means a statement or claim that a particular biologic event will, or is very likely to, occur in the future.

Progression: As used herein, the term “progression” or “cancer progression” means the advancement or worsening of or toward a disease or condition.

Proliferate: As used herein, the term “proliferate” means to grow, expand or increase or cause to grow, expand or increase rapidly. “Proliferative” means having the ability to proliferate. “Anti-proliferative” means having properties counter to or inapposite to proliferative properties.

Protein: A “protein” means a polymer of amino acid residues linked together by peptide bonds. The term, as used herein, refers to proteins, polypeptides, and peptides of any size, structure, or function. Typically, however, a protein will be at least 50 amino acids long. In some instances the protein encoded is smaller than about 50 amino acids. In this case, the polypeptide is termed a peptide. If the protein is a short peptide, it will be at least about 10 amino acid residues long. A protein may be naturally occurring, recombinant, or synthetic, or any combination of these. A protein may also comprise a fragment of a naturally occurring protein or peptide. A protein may be a single molecule or may be a multi-molecular complex. The term protein may also apply to amino acid polymers in which one or more amino acid residues are an artificial chemical analogue of a corresponding naturally occurring amino acid.

Protein expression: The term “protein expression” refers to the process by which a nucleic acid sequence undergoes translation such that detectable levels of the amino acid sequence or protein are expressed.

Purified: As used herein, “purify,” “purified,” “purification” means to make substantially pure or clear from unwanted components, material defilement, admixture or imperfection.

Regression: As used herein, the term “regression” or “degree of regression” refers to the reversal, either phenotypically or genotypically, of a cancer progression. Slowing or stopping cancer progression may be considered regression.

Sample: As used herein, the term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Signal Sequences: As used herein, the phrase “signal sequences” refers to a sequence which can direct the transport or localization of a protein.

Single unit dose: As used herein, a “single unit dose” is a dose of any therapeutic administered in one dose/at one time/single route/single point of contact, i.e., single administration event.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Split dose: As used herein, a “split dose” is the division of single unit dose or total daily dose into two or more doses.

Stable: As used herein “stable” refers to a compound that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable.

Subject: As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term means within 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time.

Synthetic: The term “synthetic” means produced, prepared, and/or manufactured by the hand of man. Synthesis of polynucleotides or polypeptides or other molecules of the present invention may be chemical or enzymatic.

Targeted Cells: As used herein, “targeted cells” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24 hr period. It may be administered as a single unit dose.

Transcription factor: As used herein, the term “transcription factor” refers to a DNA-binding protein that regulates transcription of DNA into RNA, for example, by activation or repression of transcription. Some transcription factors effect regulation of transcription alone, while others act in concert with other proteins. Some transcription factor can both activate and repress transcription under certain conditions. In general, transcription factors bind a specific target sequence or sequences highly similar to a specific consensus sequence in a regulatory region of a target gene. Transcription factors may regulate transcription of a target gene alone or in a complex with other molecules.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

The phrase “a method of treating” or its equivalent, when applied to, for example, cancer refers to a procedure or course of action that is designed to reduce, eliminate or prevent the number of cancer cells in an individual, or to alleviate the symptoms of a cancer. “A method of treating” cancer or another proliferative disorder does not necessarily mean that the cancer cells or other disorder will, in fact, be completely eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of an individual, is nevertheless deemed an overall beneficial course of action.

Tumor growth: As used herein, the term “tumor growth” or “tumor metastases growth”, unless otherwise indicated, is used as commonly used in oncology, where the term is principally associated with an increased mass or volume of the tumor or tumor metastases, primarily as a result of tumor cell growth.

Tumor Burden: As used herein, the term “tumor burden” refers to the total Tumor Volume of all tumor nodules with a diameter in excess of 3 mm carried by a subject.

Tumor Volume: As used herein, the term “tumor volume” refers to the size of a tumor. The tumor volume in mm³ is calculated by the formula: volume=(width)²×length/2.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

### EXAMPLES

### Example 1. Preparations of CEBPA-51 and MTL-CEBPA

Materials and Procedures of preparing CEBPA-saRNAs have been disclosed in WO2015/075557 and WO2016/170349 to MiNA Therapeutics Limited. The preparations of CEBPA-51 and MTL-CEBPA have been disclosed in Examples of WO2016/170349.

In brief, each strand of CEBPA-51 was synthesized on a solid support by coupling phosphoramidite monomers sequentially. The synthesis was performed on an automatic synthesizer such as an Akta Oligopilot 100 (GE Healthcare) and a Technikrom synthesizer (Asahi Kasei Bio) that delivers specified volumes of reagents and solvents to and from the synthesis reactor (column type) packed with solid support. The process began with charging reagents to the designated reservoirs connected to the reactor and packing of the reactor vessel with the appropriate solid support. The flow of reagent and solvents was regulated by a series of computer-controlled valves and pumps with automatic recording of flow rate and pressure. The solid-phase approach enabled efficient separation of reaction products as coupled to the solid phase from reagents in solution phase at each step in the synthesis by washing of the solid support with solvent.

CEBPA-51 was dissolved at ambient temperature in sodium acetate/sucrose buffer pH 4.0 and lipids were dissolved in absolute ethanol at 55° C. Liposomes were prepared by crossflow ethanol injection technology. Immediately after liposome formation, the suspension was diluted with sodium chloride/phosphate buffer pH 9.0. The collected intermediate product was extruded through polycarbonate membranes with a pore size of 0.2 μm. The target saRNA concentration was achieved by ultrafiltration. Non-encapsulated drug substance and residual ethanol were removed by subsequent diafiltration with sucrose/phosphate buffer pH 7.5. Thereafter, the concentrated liposome suspension was 0.2 μm filtrated and stored at 5±3° C. Finally, the bulk product was formulated, 0.2 μm filtrated and filled in 20 ml vials.

MTL-CEBPA was presented as a concentrate solution for infusion. Each vial contains 50 mg of CEBPA-51 (saRNA) in 20 ml of sucrose/phosphate buffer pH about 7.5.

### Example 2. CEBPA-saRNA Increased White Blood Cells and Neutrophil Levels

In this example, the effect of CEBPA-saRNA on WBC and neutrophils (NEU) levels was studied.

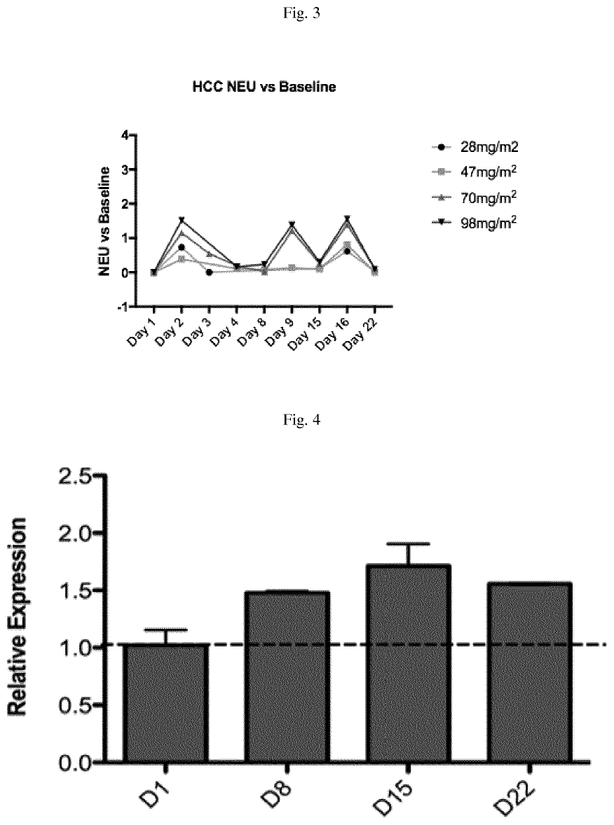

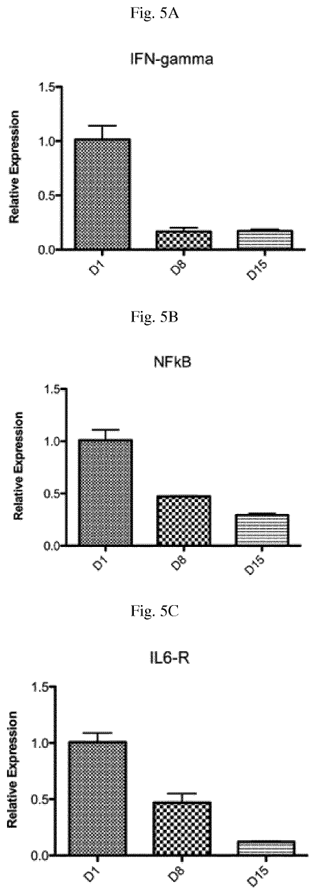

MTL-CEBPA was dosed on Day 1, Day 8, and Day 15 at 28 mg/m², 47 mg/m², 70 mg/m², or 98 mg/m². The WBC and neutrophils (NEU) levels of patients were monitored over time after MTL-CEBPA treatment. As shown in FIG. 2 and FIG. 3, an increase of WBC and an increase of NEU occurred after each dose.

### Example 3. CEBPA-saRNA Increased CEBPA-mRNA Levels in WBC and Reduced Inflammation

In this example, the effect of CEBPA-saRNA on inflammatory markers was studied.

MTL-CEBPA was dosed on Day 1, Day 8, and Day 15. Blood samples were collected on Day 1 (pre-dose), Day 2, Day 8 (pre-dose) and Day 15 (pre-dose). 6 mL of patient's whole blood was collected in a K2 vacutainer (lavender/purple cap); the tube already containing a measured amount of anti-coagulant. The spike cap from the top end of the preassembled LeukolLOCK filter unit was removed. The blood collected in the 6 mL K2 vacutainer passed through the filter and was transferred to a 9 ML additive free vacutainer. 3 mL PBS and then 3 mL RNALater were injected through the filter. The vacutainer was then sealed.

| | | Day 1 | | Day 8 | Day 15 |
|---|---|---|---|---|---|
| Procedure | Screening | Pre-dose | Day 2 | Pre-dose | Pre-dose |
| mRNA | x | x | x | x | x |

6 mL of patient's whole blood was collected in a K2 vacutainer (lavender/purple cap); the tube already containing a measured amount of anti-coagulant. The spike cap from the top end of the preassembled LeukoLOCK™ filter unit was removed. The blood collected in the 6 mL K2 vacutainer passed through the filter and was transferred to a 9 ML additive free vacutainer. 3 mL PBS (Phosphate Buffered Saline) and then 3 mL RNALater® stabilization solution were injected through the LeukoLOCK™ filter. The filter unit was saturated with RNALater®. The white blood cells (WBCs) captured in the filter unit were used for downstream studies.

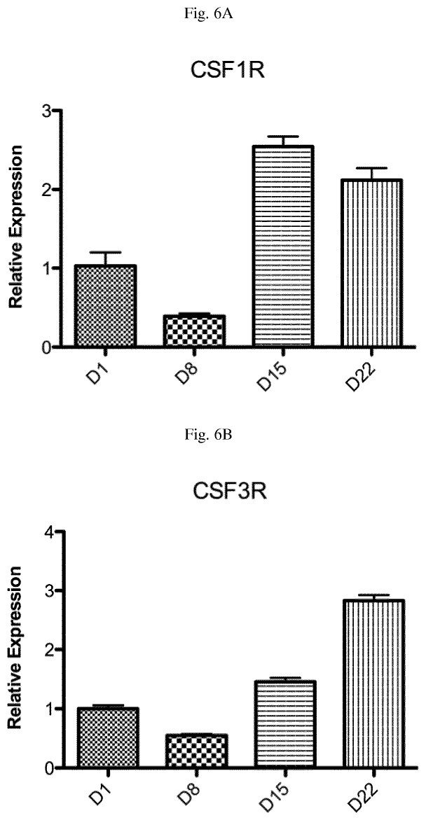

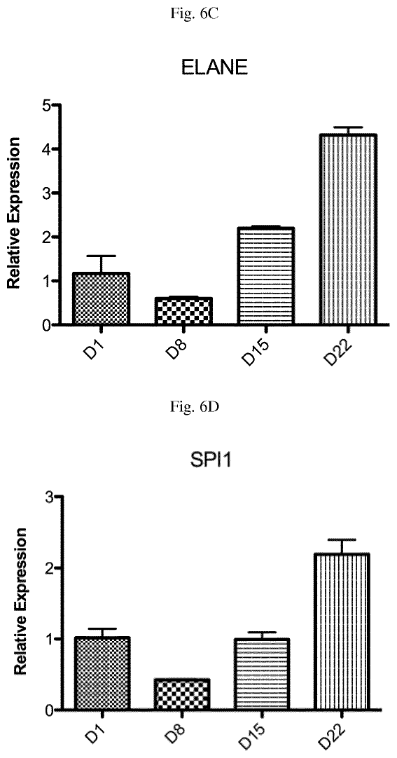

mRNA in the WBCs was extracted. qPCR was carried out to determine the relative levels of genes using appropriate qPCR probes. CEBPA-mRNA changes are shown in FIG. 4. CEBPA-mRNA increased to 1.47 fold by Day 8. Maximum increase in the patient was observed at Day 15 at 1.7 fold. At Day 22, a 1.5 fold increase was maintained. The changes in inflammatory markers are shown in FIGS. 5A (IFN-gamma), 5B (NFkB), and 5C (IL6-R). A reduction in inflammatory markers in WBCs was observed on Day 8 (7 days after dose 1), and Day 15 (7 days after dose 2). Therefore, MTL-CEBPA suppressed inflammation.

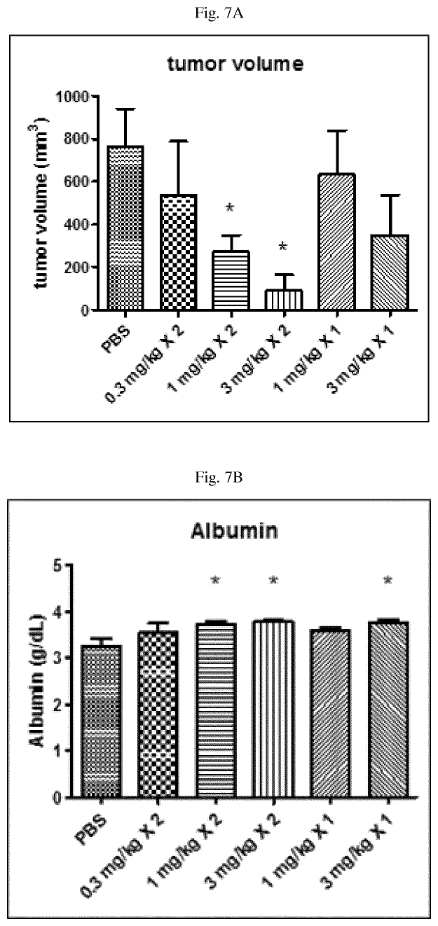

The changes in the downstream CEBPA targets are shown in FIG. 6. An increase in CSF1R (FIG. 6A), CSF3R (FIG. 6B), ELASE (FIG. 6C) and SPI1 (FIG. 6D) levels were observed.

### Example 4. CEBPA-saRNA Dosing Regimen Studies

In this example, the effect of CEBPA-saRNA dosing regimen on improving liver function in the CCL4 model of liver fibrotic disease was studied. CCL4 model of rats with liver damage and fibrosis were treated with 2 doses of MTL-CEBPA spaced 48 hours apart (Day 1 and Day 3) or spaced 9 days apart (Day 1 and Day 10). MTL-CEBPA had a bigger effect on improving liver function for rats treated with MTL-CEBPA on Day 1 and Day 3.

Male SD rats were administered for 8 weeks with CCL4 to induce liver damage and fibrosis. Levels of Alanine Aminotransferase (ALT) were measured as a marker of liver damage.

Initially MTL-CEBPA (1 mg/kg) was administered on Day 1 and Day 10 and this treatment schedule did not reduce the ALT levels. After 2 weeks (Day 15) ALT levels had increased by 26%. ALT levels of rat groups were shown below.

TABLE 4-1
ALT levels of rat groups

| Treatments | ALT levels |
|---|---|
| CCL4 + MTL- | Week 8 −207 +/− 25 U/ml, Week 10 −267 +/− 29 U/ml |
| Fluc control | |
| CCl4 + | Week 8 −204 +/− 21 U/ml, Week 10 −258 +/− 24 U/ml |
| MTL-CEBPA | |

Rats that had been on CCL4 for 11 weeks and had increased ALT levels at time of treatment were then given MTL-CEBPA (1 mg/kg) on Day 1 and Day 3 to explore if two more closely spaced doses would improve activity. Surprisingly treatment with 2 doses of MTL-CEBPA on Day 1 and Day 3 caused a 43% reduction in ALT after 2 weeks supporting a therapeutic benefit with this regimen that was not seen if the same total dose was spaced approximately a week apart. ALT levels of rat groups were shown below.

TABLE 4-2
ALT levels of rat groups

| Treatments | ALT levels |
|---|---|
| CCl4 + MTL | Week 11 −301 +/− 56 U/ml, Week 13 −284 +/− 20 U/ml |
| Fluc Control | |
| CCL4 − | Week 11 −328 +/− 81 U/ml, Week 13 −188 +/− 11 U/ml |
| MTL- | |
| CEBPA | |

With either dosing schedule, the MTL-FLuc control had no impact on reducing ALT levels, demonstrating this effect was specific for the CEBPA51 encapsulated in the SMARTICLES® nanoparticles.

### The Impact of a Single or Two Split Doses of MTL-CEBPA

A series of further studies were conducted in normal rats investigating the effect of different dosing regimens on CEBPA-mRNA levels in the liver. The relative levels of CEBPA-mRNA in the livers were measured by qPCR.

A single dose of MTL-CEBPA (3 mg/kg) caused a transient rise in CEBPA-mRNA in the liver at 8 hrs that had fallen significantly by 24 hrs and after 7 days had returned to near normal levels.

Splitting the 3 mg/kg single dose into two doses of 1.5 mg/kg lead to an increased rise in liver CEBPA-mRNA levels. When 2 doses of 1.5 mg/kg MTL-CEBPA were given on Day 1 and Day 2, CEBPA-mRNA levels in the liver 24 hrs after the second dose were increased to a greater extent than 24 hrs after a dose of 3.0 mg/kg MTL-CEBPA. When 2 doses of 1.5 mg/kg MTL-CEBPA were given on Day 1 and Day 3, CEBPA-mRNA levels in the liver 24 hrs after the second dose were increased to a greater extent than 24 hrs after a dose of 3.0 mg/kg MTL-CEBPA and were still significantly increased 7 days after the last dose in contrast to the single dose group where CEBPA-mRNA levels had returned to near normal after 7 days.

TABLE 4-3
qPCR mRNA levels of CEBPA relative to PBS control in
liver homogenates

| | CEBPA-mRNA by qPCR relative | | |
|---|---|---|---|
| | to PBS control group | | |
| Dose and time interval | 8 hr | 24 hr | 1 Week |
| 3 mg/kg Day 1 only | 2.43 | 1.14 | 1.10 |
| 1.5 mg/kg Day 1, Day 2 | ND | 1.47 | ND |
| 1.5 mg/kg Day 1, Day 3 | ND | 1.25 | 1.65 |

ND—not done

These studies indicate that giving closely spaced split doses, either 24 hours apart (Day 1 & Day 2) or 48 hours apart (Day 1 & Day 3), compared to a single dose and/or doses spaced approximately a week apart, leads to increased levels of CEBPA-mRNA at 24 hrs and importantly a sustained activation that last at least 7 days post the initial dose. The increased and sustained levels of CEBPA-mRNA using the split dose regimen are consistent with the improved activity of this split dose regimen in the CCL4 model.

In another study in normal rats the advantage of splitting the dose and dosing 48 hrs apart was confirmed (Table 4-4). Also, further splitting the single dose into 3 doses given 24 hrs apart resulted in an even bigger increase on CEBPA-mRNA levels in the liver 48 hrs and 7 days after the last dose (Table 4-4) compared to the single dose.

TABLE 4-4
qPCR mRNA levels of CEBPA in liver after splitting single dose
into 2 or 3 doses

| Dose and timing | CEBPA mRNA day 2* | CEBPA mRNA day 7* |
|---|---|---|
| 3 mg/kg Day 1 | 1.14 +/− 0.09 (NS) | 1.37 +/− 0.28 (NS) |
| 1.5 mg/kg Day 1, | 1.50 +/− 0.11 (p = 0.008) | 1.59 +/− 0.19 (p = 0.034) |
| Day 3 | | |
| 1 mg/kg Day 1, | 1.92 +/− 0.32 (P = 0.05) | 1.99 +/− 0.33 (p = 0.039) |
| Day 2, Day 3 | | |

*mRNA levels by qPCR relative to PBS control group

Therefore, it has been found, surprisingly, with the same amount of total dosing, 3 split dosings given on Day 1, Day 2, and Day 3 work better than 2 split dosings given on Day 1 and Day 3, which is better than a single dose given on Day 1.

### DEN Study—Single vs Biweekly Dosing of MTL-CEBPA

In this pre-clinical study, diethylnitrosamine (DEN) model of liver cancer was used. Briefly DEN was administered for 9 weeks in the drinking water as reported previously in male Wistar rats followed by a 2-week wash-out period. At week 10-11, animals were randomized for each experimental arm. Tumor-bearing animals were treated with varying doses of MTL-CEBPA given as a single weekly or biweekly (48 hrs apart) dose for 2 weeks and the effects were compared to a PBS control group. The tumour burden at the end of the experiment was assess by measuring the diameters of all of the macroscopically visible nodules on the liver surface and in 5-mm sliced sections of the liver.

The dose groups in the study were:

| Group | Dose (mpk) | Dose per week | Weekly dose (mpk) |
|---|---|---|---|
| 1 | PBS | PBS | PBS |
| 2 | 0.3 | 2 | 0.6 |
| 3 | 1.0 | 2 | 2.0 |
| 4 | 3.0 | 2 | 6.0 |
| 5 | 1.0 | 1 | 1.0 |
| 6 | 3.0 | 1 | 3.0 |

There were 5-6 tumour bearing animals/group.

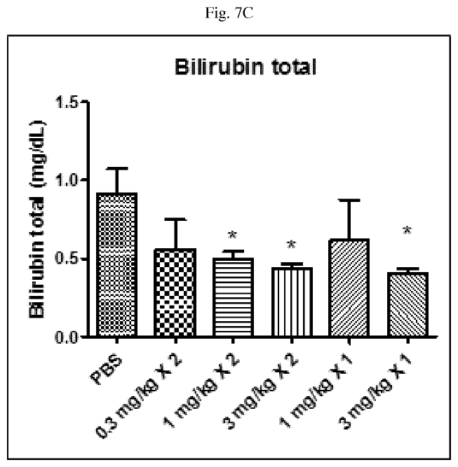

As shown in FIG. 7A, there was a dose response for tumour growth inhibition across the weekly dose range of 0.6 to 6.0 mpk in the biweekly dosing schedule with 1 mg/kg and 3 mg/kg×2 dose levels reaching statistical significance. In the 3 mg/kg biweekly dose 2/6 of the animals had no apparent tumour burden at the end of study vs all 6 control animals had tumour burden exceeding 100 mm³.

There was also a dose response with the single dose schedule but neither 1 or 3 mg/kg led to significant anti-tumour activity.

Comparing groups 3 that received a total dose of 2 mg/kg MTL-CEBPA as a split dose with group 6 that received a total dose of 3 mg/kg as a single dose, it does appear from this study there is benefit in giving a split dose in terms of anti-tumour efficacy.

As shown in FIG. 7B and FIG. 7C, there was a much milder dose response on liver function tests across weekly dose range with the higher dose levels in the single and split regimes leading to a small significant increase in serum albumin and approximately a 50% drop in serum bilirubin. The 3 most active doses in the anti-tumour component of the study produced the biggest and significant effects on albumin and bilirubin. This limited dose response on liver function is consistent with CCl4 fibrosis/cirrhosis studies in rats where a limited dose response across 0.3-3 mg/kg dose levels was observed on increasing albumin and decreasing bilirubin levels.

Overall the data support exploring a biweekly split dose schedule in the clinic for treating HCC and are consistent with increased CEBPA mRNA liver PD with split dosing vs single dose in the Epistem normal rat studies.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

1. A method of increasing white blood cell (WBC) or neutrophils (NEU) levels comprising administering a synthetic isolated saRNA which up-regulates the expression of C/EBPα gene, wherein the saRNA comprises a strand with a sequence that is at least 80% complementary to a region on SEQ ID No. 3, and wherein the strand has 14-30 nucleotides.

2. The method of claim 1, wherein the saRNA comprises a strand with SEQ ID No. 1.

3. The method of claim 1, wherein the saRNA is double-stranded and comprises an antisense strand of SEQ ID No. 1 and a sense strand of SEQ ID No. 2.

4. The method of claim 1, wherein the saRNA is encapsulated into liposomes.

5. The method of claim 4, wherein the liposome comprises 1-palmitoyl-2-oleoyl-sn-glycero-3-phosphocholine (POPC), 1,2-dioleoyl-sn-glycero-3-phosphoethanolamine (DOPE), cholesteryl-hemi succinate (CHEMS), and 4-(2-aminoethyl)-morpholino-cholesterol hemisuccinate (MOCHOL).

6. The method of claim 5, wherein the molar ratio of POPC:DOPE:CHEMS:MOCHOL is around 6:24:23:47.

7. The method of claim 4, wherein the size of the liposome is between about 50 nm to about 150 nm or between about 100 nm to about 120 nm.

8. The method of claim 1, wherein WBC count or neutrophil count increases at least 10%, 20%, 30%, 40%, 50%, 60%, 70%, 80%, 90%, 100%, 110%, 120%, 130%, 140%, 150%, two folds, three folds, four folds, five folds, or ten folds compared to the WBC count or neutrophil count before saRNA administration.

9. A method of reducing inflammation of a cell comprising administering a synthetic isolated saRNA which up-regulates the expression of C/EBPα gene, wherein the saRNA comprises a strand with a sequence that is at least 80% complementary to a region on SEQ ID No. 3, and wherein the strand has 14-30 nucleotides.

10. The method of claim 9, wherein the saRNA comprises a strand with SEQ ID No. 1.

11. The method of claim 9, wherein the saRNA is double-stranded and comprises an antisense strand of SEQ ID No. 1 and a sense strand of SEQ ID No. 2.

12. The method of claim 9, wherein the saRNA is encapsulated into liposomes.

13. The method of claim 12, wherein the liposome comprises 1-palmitoyl-2-oleoyl-sn-glycero-3-phosphocholine (POPC), 1,2-dioleoyl-sn-glycero-3-phosphoethanolamine (DOPE), cholesteryl-hemisuccinate (CHEMS), and 4-(2-aminoethyl)-morpholino-cholesterol hemisuccinate (MOCHOL).

14. The method of claim 13, wherein the molar ratio of POPC:DOPE:CHEMS:MOCHOL is around 6:24:23:47.

15. The method of claim 12, wherein the size of the liposome is between about 50 nm to about 150 nm or between about 100 nm to about 120 nm.

16. The method of claim 1, wherein the cell is a white blood cell.

17. The method of claim 16, wherein the interferon gamma (IFN-gamma), nuclear factor kappa-light-chain-enhancer of activated B cells (NFkB), and/or interleukin 6 receptor (IL6-R) levels are reduced.

18. The method of claim 17, wherein the level of IFN-gamma, NFkB, or IL6-R decrease at least 10%, 20%, 30%, 40%, 50%, 60%, 70%, or 80% compared to the level of IFN-gamma, NFkB, or IL6-R before saRNA administration.

19. A method of up-regulating CEBPA gene expression in a subject comprising administering a double-stranded saRNA comprises an antisense strand of SEQ ID No. 1 and a sense strand of SEQ ID No. 2, wherein the subject receives at least two doses of the saRNA, wherein the doses are 24 hours or 48 hours apart.

20. The method of claim 19, wherein the saRNA is encapsulated into liposomes.

21. The method of claim 20, wherein the liposome comprises 1-palmitoyl-2-oleoyl-sn-glycero-3-phosphocholine (POPC), 1,2-dioleoyl-sn-glycero-3-phosphoethanolamine (DOPE), cholesteryl-hemi succinate (CHEMS), and 4-(2-aminoethyl)-morpholino-cholesterol hemisuccinate (MOCHOL).

22. The method of claim 21, wherein the molar ratio of POPC:DOPE:CHEMS:MOCHOL is around 6:24:23:47.

23. The method of claim 19, wherein the CEBPA gene expression in the liver of the subject is increased.

24. The method of claim 19, wherein the subject has a liver disease.

25. The method of claim 24, wherein the subject has liver fibrosis.

26. The method of claim 19, wherein the subject receives 2 doses 48 hours apart.

27. The method of claim 19, wherein the subject receives 3 doses 24 hours apart.


SEQUENCE LISTING (3 sequences)

SEQ ID NO: 1 - RNA, 21 nt, Artificial Sequence
  Description of Artificial Sequence Synthetic oligonucleotide
  modified_base (20)..(21): 2′-OMe modified nucleotide
gaccagugacaaugaccgcuu

SEQ ID NO: 2 - RNA, 21 nt, Artificial Sequence
  Description of Artificial Sequence Synthetic oligonucleotide
  modified_base (1)..(1): Inverted abasic modification
  modified_base (1)..(2): 2′-OMe modified nucleotide
  modified_base (4)..(4): 2′-OMe modified nucleotide
  modified_base (10)..(10): 2′-OMe modified nucleotide
  modified_base (14)..(14): 2′-OMe modified nucleotide
  modified_base (20)..(21): 2′-OMe modified nucleotide
gcggucauugucacuggucuu

SEQ ID NO: 3 - DNA, 4001 bp, Homo sapiens
tccctctcccaccaggggtatacatcctcagagctgacccacgacctagctttctggtgtgactcggggtgggggctcccactggtcacctggtgacccccatcgcagtgagttccgccccaaggggaagcccagcctatagcaggctggggtggggtgtgtgcggagggaggtgggagaggcgtggaactagagaccctccaccttcatgtagaactaggggaacaaccttaggttccaagccccaagtccctatgtttccacccctttctaaggacaggcgtggaggagcggctggggctggcgggcttgtcgggatctcagctccctgagccctcctcctgccacgggcctgctcccctccttctctcatgggggtctgctgtagcctcgggaaggaggcaggaaacctccaaataaaatgacaaggcacgatttgctccccctactcagtaggcattggagcggtgagtttgcatttccaaggcacaaggttatcctaaatactagagttgccgggctcccagctcagccccaagaattctcccctcctcgcagggagaagccaccgcctggccccctcatcttagacgcaccaagtccggcgcagaggaagggaggggacacgcggagcaggccaggctttcaggaggcaccggaatctcctagtcctggctcgcacggctcgggcaagcctcgagatccggcgaccccaaaccactccctgggtccccgccggaggctggcccagggcggtcccacagccgcgcgcctcacgcgcagttgcccatggccttgaccaaggagctctctggcagctggcggaagatgccccgcagcgtgtccagttcgcggctcagctgttccacccgcttgcgcaggcggtcattgtcactggtcagctccagcaccttctgctgcgtctccacgttgcgctgcttggccttgtcgcggctcttgcgcaccgcgatgttgttgcgctcgcgccgcacccggtactcgttgctgttcttgtccaccgacttcttggccttgcccgcgccgctgccgccactcgcgcggaggtcggggtgcgcggcgcccagccccttgagcgcgctgccagggcccggcaggccggcggcaccgagcgcgggcgcggggtgcgggctgggcacgggcgtgggcggcggcgtggggtgaccgggctgcaggtgcatggtggtctggccgcagtgcgcgatctggaactgcaggtgcggggcggccaggtgcgcgggcggcgggtgcgggtgcgggtgcgagggcggcggcggcggcggcggctggtaagggaagaggccggccagcgccagctgcttggcttcatcctcctcgcggggctcctgcttgatcaccagcggccgcagcgccggcgccccgacgcgctcgtacaggggctccagcctgccgtccaggtagccggcggccgcgcagccgtagccgggcgggggcccgtgcgctcccccgggcatgacggcgccgccggggcccgcgggcgcgcccgggtagtcaaagtcgccgccgccgccgccgcccgtggggcccacggccgccttggccttctcctgctgccggctgtgctggaacaggtcggccaggaactcgtcgttgaaggcggccgggtcgatgtaggcgctgatgtcgatggacgtctcgtgctcgcagatgccgcccagcggctccggggcggcaggtggggcgggaggctgcgcggggcccgcgccccggggaaagccgaaggcggcgctgctgggcgcgtgcggggggctctgcaggtggctgctcatcgggggccgcggctccgcctcgtagaagtcggccgactccatgggggagttagagttctcccggcatggcgagcctcggcggcctccagcctgcgcggggcgtcgccgccgcccacccggagaccctgctcgcccgcgcccgcgcacctccgggtcgcgaatggcccggcccgcgccggcccagcttttatacccggcaggccgcgtcgccccctagagtccgaggcggcctctgtccccgggctgcggcggcgcggcgcctgctgggtcctagcgcgcggccggcatggggcggcgaaccagcgcggcacagcgccgcgctccccaggcaggccgcggcgcaacgcccaccgcctccagcgcgcccagcagagccgcggcgctcgctccaagctccgcccccggcccggccgtcgcccccgcgcccacgtggtcggtagcgggggccccctcctcctgcctgccctaggcgcccgtatccagccacggccgggagcccaggagtatcccgaggctgcacggggtaggggtggggggcggagggcgagtcttggtcttgagctgctggggcgcggattctctttcaaagccagaaccaggcctgtcccggacccgcgtcccggggaggctgcagcgcagagcagcggggctggggccggtggggggccgtttgggacgcgcggagaggtcctgagcgcggtggctctgcgtctcctagctctgatctccaggctacccctgtgattccgcgcagaggtacctctcggaggacgccggggtcccatgggcggcgccgcgcagggcgctaggaccccgcggggagcggaggcggcctcggcccgggagcctggaggacctggccggtcgatccgcccgggctggaaaactttctttataattacttctccaggtcggagcgcgcggcttgctaggcgcgcggggccggcgctgttacccggcgtggagtcgccgattttttttcctgcgggaccgcggggccccccagactagcggagctggacgccggggcgagcacggggaggggcgcaccgagggaggagacaaacttaactctggggccgggattccgaggcgggggccgcagccctcgaggcccgaagccaccgcttcctcccccgcctccccattcaggtgggcgccaacggcgggagcgagggtgtccaggccgccgggctgccaggtccgagcacgcacagggagaactctgcccagtggttcgccgggcgctgtagtccccgggatcctagggaccgaggcggccaggccctggggccccttgagtgcggcagctaatgctctcaccgcggcgggggaaggagcttgccaccgagacccccagccacgtgcgtccctcgcattctttaccggggccggggtggcggctacggaccgtcagctgggcccagatggagtcttgggagccctcaagtgtctcctgtccttgcccgcgccgcccctcgccactggcgctgaggcctgacgccgcctgcgtcccggctagaggcgcgcttgcctacaggtgagggaagacccccttcaccgacagtggccttaggcctggcaaggcgccacgacccgcccaggagccccggagggggcacagctaaaaacaccgctggagagccccgagcttccacgacgatcgcagtaaagaagcagtttcatctgggcaacgcacactgcgctttaatcaagttcctattcaacatagtcccagtgattaatagcccaactgcttcgttttcggtccagagctcataaacaagatatttttagcttgacgcttttggacgggagggagtaaaaaccagatacgttaaataaatatcccgatgtgagccggagagctgcttgctgagccaaatgcaggacccattcatatagcattcacctgtggagggagacctggacggaaatcaaaaagcaccaagagcgatttgcgtttttttctgcggtgctaaaactaatggcttttcctacctaggaacaaagaaacgccactgtacatgcacggttcccggcctgtggagttgtgggaggaaggcgatgtctggccttttttgcacagctgctgttgcctgcccagagatcgggaactctgccccgtaggactggaagaaacctcagtaatgggaataagactttgtccaatagggggctgatgaatgtgtg